(19) United States
(12) Patent Application Publication
(10) Pub. No.: US 2025/0082813 A1
(43) Pub. Date: Mar. 13, 2025

(54) Air Treatment System

(71) Applicant: Randall Franklin Roe, Clearwater, FL (US)

(72) Inventor: Randall Franklin Roe, Clearwater, FL (US)

(21) Appl. No.: 18/531,275

(22) Filed: Dec. 6, 2023

Related U.S. Application Data
(60) Provisional application No. 63/582,136, filed on Sep. 12, 2023.

(51) Int. Cl. A61L 9/14; A61L 9/18; F24F 3/16; F24F 8/22; F24F 8/24; F24F 8/50; F24F 11/61

(52) U.S. Cl. CPC A61L 9/14; A61L 9/18; F24F 3/16; F24F 8/22; F24F 8/24; F24F 8/50; F24F 11/61; A61L 2209/11; A61L 2209/12; A61L 2209/134; A61L 2209/16

(57) ABSTRACT

An air treatment system is a system that sanitizes, disinfects, and/or deodorizes a building's air-conditioned spaces to improve the indoor air quality. The present invention is designed to sanitize, disinfect, and/or deodorize the building's areas through which conditioned air flows. The system includes a timer relay, at least one pump, a plurality of atomizer assemblies, a treatment solution, and a solution reservoir. The timer relay allows for the periodic application of the treatment solution onto the building's target areas. The at least one pump allows for the controlled supply of the treatment solution from the solution reservoir to the plurality of atomizer assemblies. The plurality of atomizer assemblies allows the safe application of the treatment solution in an atomized form. The treatment solution includes various compositions that safely improve the indoor air quality. The solution reservoir securely retains and facilitates the dispensing of the treatment solution during the system's operation.

### FIELD OF THE INVENTION

The present invention relates generally to Heating, Ventilation, and Air Conditioning (HVAC) systems and cleaning, disinfecting, and/or deodorizing systems. More specifically, the present invention provides a system that cleans, disinfects, and/or deodorizes a building's air-conditioned spaces by administering air treatments into the facility's HVAC system or directly into the air-conditioned spaces.

### BACKGROUND OF THE INVENTION

In recent years, indoor air quality has worsened as buildings have become more energy efficient. Recent studies have shown that indoor air has a significantly higher number of pollutants than outdoor air. In some studies, indoor air has been shown to have up to five times more pollutants than outdoor air. Major indoor air pollution sources are the pollutants that grow in the building's Heating, Ventilation, and Air Conditioning (HVAC) system in addition to the pollutants that are brought inside by different means. For example, studies have shown that bacteria such as mold spores grow inside the building's supply air ductwork, return air ductwork, evaporator coil encasements, and/or surrounding surfaces. Various indoor air filtration products such as media filters and electronic air filters are currently available to help improve indoor air quality. However, these products are inefficient at killing bacteria and/or eliminating pollutants that grow in the supply air ductwork, return air ductwork, evaporator coil encasements, and/or on surrounding surfaces of the building's HVAC system. Thus, there is a need for a better solution that improves indoor air quality.

An objective of the present invention is to provide an air treatment system that cleans, disinfects, and/or deodorizes the indoor air supplied by a building's HVAC system. The present invention improves the indoor air quality by atomizing a liquid solution that is applied to different areas of the building's HVAC system including, but not limited to, the supply air ductwork, the return air ductwork, the evaporator coils, etc. Another objective of the present invention is to provide an air treatment system that can directly treat the indoor air present at different locations inside the building. The present invention can also directly atomize the liquid solution into the building's air-conditioned spaces. Another objective of the present invention is to provide an air treatment system that can be configured for automatic operation with the building's HVAC system. The present invention can be configured for periodic operation that may coincide with the operation of the building's HVAC system or another building's system. The present invention may also be configured to operate independent from the operation of the building's HVAC system. Additional features and benefits of the present invention are further discussed in the sections below.

### SUMMARY OF THE INVENTION

The present invention provides an air treatment system that cleans, disinfects, and/or deodorizes indoor air-conditioned spaces via periodic treatment applications of a treatment solution into the building's Heating, Ventilation, and Air Conditioning (HVAC) system. The present invention, also referred to as “AirRoe Pure,’ allows treatment solutions to be applied to the HVAC system's supply and/or return air ductwork, evaporator coil encasements, etc. Further, the treatment solution composition can include, but is not limited to, an amount of disinfectant, an amount of deodorizer, an amount of fragrance, etc., or a combination of ingredients thereof. Different compositions of the treatment solution can be utilized by the user to meet the user's needs. Further, the present invention can be configured to treat air-conditioned spaces not containing ductwork or blower-motor-based operations (e.g., libraries, schools, and other public/private buildings not using forced air heating and/or air conditioning). To do so, the air treatment system can be provided as a separate system that can be retrofitted to the building's structure to treat the appropriate air-conditioned spaces. The air treatment system can also be integrated into the building's HVAC system to be part of the HVAC system. Further, the air treatment system can also include means to directly apply the treatment solution into the target building's air-conditioned spaces. The present invention can also apply fragrances and/or other solutions safe to people. Further, the air treatment system can be configured to operate in an automated manner so that the treatment solution can be applied periodically or in conjunction with the operation of the building's HVAC system. Furthermore, the air treatment system can also be configured to operate with other external systems installed in the target building.

### BRIEF DESCRIPTIONS OF THE DRAWINGS

FIG. 1 is a schematic view of the present invention, wherein the atomizer assemblies of the present invention are shown with atomizer nozzles.

FIG. 2 is a schematic view of the present invention, wherein the atomizer assemblies of the present invention are shown with fogger nozzles.

FIG. 3 is a schematic view of the electronic connections and the electrical connections of a first embodiment of the present invention, wherein the present invention is shown with a controller and a power source, wherein the electronic connections are shown with dot-dashed lines, and wherein the electrical connections are shown with solid lines.

FIG. 4 is a schematic view of the electronic connections and the electrical connections of a second embodiment of the present invention, wherein the electronic connections are shown with dot-dashed lines, and wherein the electrical connections are shown with solid lines.

FIG. 5 is a schematic view of the present invention, wherein a composition of the treatment solution is shown.

FIG. 6 is an exploded perspective view of a first embodiment of the atomizer assembly of the present invention.

FIG. 7 is a perspective view of the first embodiment of the atomizer assembly of the present invention.

FIG. 8 is a schematic view of the present invention, wherein an atomizer assembly of the present invention is shown installed in a duct.

FIG. 9 is a schematic view of the solution reservoir of the present invention, wherein the solution reservoir is shown with a heating element.

### DETAIL DESCRIPTIONS OF THE INVENTION

All illustrations of the drawings are for the purpose of describing selected versions of the present invention and are not intended to limit the scope of the present invention.

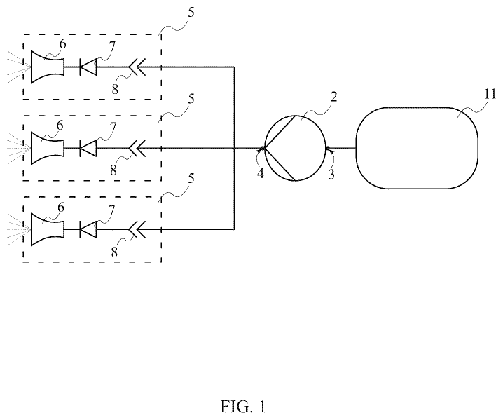

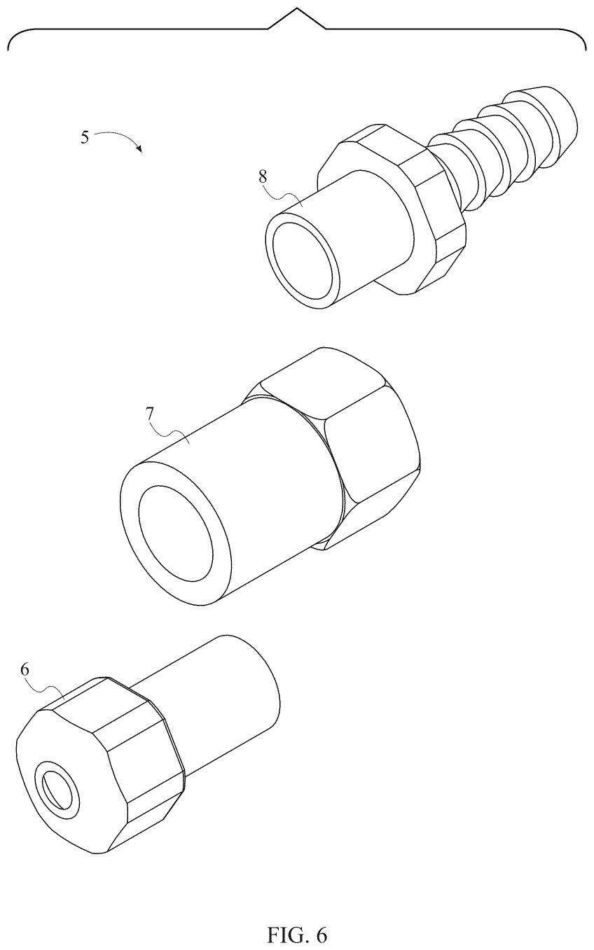

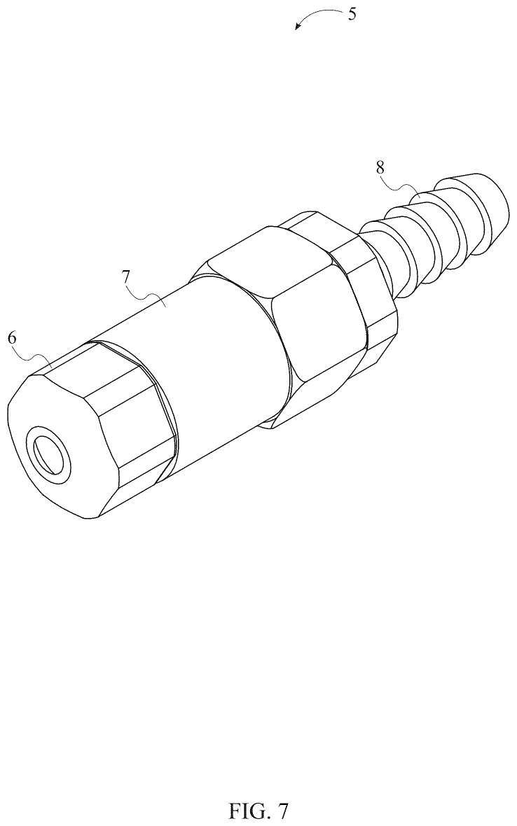

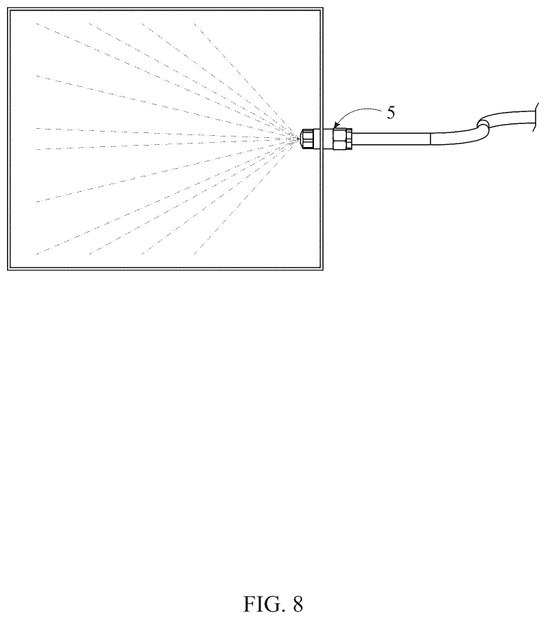

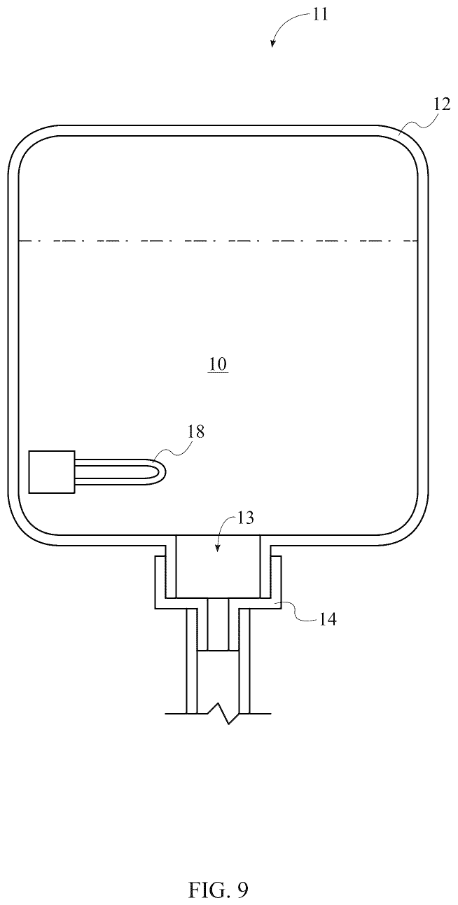

The present invention provides an air treatment system that sanitizes, disinfects, and/or deodorizes a building's air-conditioned spaces to improve the indoor air quality. The present invention is designed to sanitize, disinfect, and/or deodorize the areas through which conditioned air flows. For example, the present invention can sanitize, disinfect, and/or deodorize different areas of the building's Heating, Ventilation, and Air Conditioning (HVAC) system including, but not limited to, the supply air ductwork, return air ductwork, and/or evaporator coils through time-controlled applications during heating and cooling cycles. In addition, the present invention can directly treat the building's air-conditioned spaces without the use of external system. This way, the present invention can kill various bacteria and viruses including, but not limited to, bactericidal, virucidal, fungicidal, tuberculocidal, HIV-1 (AIDS virus), Influenza A2, Avian Flu, Aspergillus niger (black mold), Escherichia coli, and odors. As can be seen in FIG. 1 through 9, the present invention comprises a timer relay 1, at least one pump 2, a plurality of atomizer assemblies 5, a quantity of treatment solution 10, and a solution reservoir 11. The timer relay 1 allows for the periodic application of the quantity of treatment solution 10 onto the target areas. The at least one pump 2 allows for the controlled supply of the quantity of treatment solution 10 from the solution reservoir 11 to the plurality of atomizer assemblies 5. The plurality of atomizer assemblies 5 allows the application of the quantity of treatment solution 10 in an atomized form. The quantity of treatment solution 10 can include various solution compositions that safely improve the indoor air quality. The solution reservoir 11 securely retains the quantity of treatment solution 10 during the operation of the present invention.

The general configuration of the aforementioned components reduces pollutants present inside the building's structure to improve the indoor air quality. The present invention is preferably designed to automatically operate under predetermined conditions so that the quantity of treatment solution 10 can be periodically applied to the target areas. As can be seen in FIG. 1 through 9, the at least one pump 2 comprises a pump inlet 3 and a pump outlet 4. The pump inlet 3 allows the flow of the quantity of treatment solution 10 into the at least one pump 2, while the pump outlet 4 allows the flow of the pressurized quantity of treatment solution 10 out of the at least one pump 2. Further, the quantity of treatment solution 10 is retained within the solution reservoir 11 until the quantity of treatment solution 10 is supplied to the at least one pump 2. So, the solution reservoir 11 is in fluid communication with the pump inlet 3 to allow the inflow of the quantity of treatment solution 10 into the at least one pump 2 from the solution reservoir 11. Further, each of the plurality of atomizer assemblies 5 is in fluid communication with the pump outlet 4 so that appropriate amounts of the pressurized quantity of treatment solution 10 can flow towards the plurality of atomizer assemblies 5. The plurality of atomizer assemblies 5 atomizes the pressurized quantity of treatment solution 10 that enable an efficient application of the quantity of treatment solution 10 in an atomized form on the target surfaces. Further, the timer relay 1 is electronically connected to the at least one pump 2 to facilitate the periodic operation of the at least one pump 2, which enables the periodic application of the quantity of treatment solution 10. In the preferred embodiment, the present invention can be configured to operate according to the building's requirements or the user's needs. For example, microbial testing can be performed prior to the system installation to quantify and qualify the types and levels of bacteria and/or pollutants present inside the building. According to the testing results, the proper operational settings of the timer relay 1 can be determined to eliminate or minimize pollutants and/or bacteria specific to each air-conditioned space. Afterwards, adjustments to the periodic air treatments can be made to the timer relay 1 to shorten or lengthen treatment cycle times, thereby optimizing the level of actual system performance results to equal or exceed the desired level of air and surface pollutants and/or bacteria.

As previously discussed, the present invention can be designed to apply pressurized quantity of treatment solution 10 in different forms in the desired building areas. In one embodiment, each of the plurality of atomizer assemblies 5 may comprise an atomizer nozzle 6, a check valve 7, and a tubing adapter 8, as can be seen in FIG. 1. The atomizer nozzle 6 allows for the pressurized quantity of treatment solution 10 to be applied in an atomized form onto the target areas. The check valve 7 and the tubing adapter 8 allows for the secure connection of the atomizer nozzle 6 to the at least one pump 2 via the appropriate tubing. So, the atomizer nozzle 6 is terminally connected to the check valve 7 to secure the atomizer nozzle 6 to the check valve 7. Similarly, the tubing adapter 8 is terminally connected to the check valve 7, opposite to the atomizer nozzle 6, to secure the check valve 7 to the tubing adapter 8. Further, the tubing adapter 8 is in fluid communication with the pump outlet 4 to allow the flow of the pressurized quantity of treatment solution 10 from the at least one pump 2 to the corresponding atomizer nozzle 6. This way, each of the plurality of atomizer assemblies 5 applies the quantity of treatment solution 10 in an atomized form onto the target areas.

Various appropriate tools and fastening methods can be used to secure each of the plurality of atomizer assemblies 5 to the target area, such as different areas of the building's HVAC system. For example, in one embodiment, each check valve 7 is threaded into the corresponding tubing adapter 8 and tightened to avoid leakage. A plastic ring grooved locking seal cap is engaged with the tubing adapter 8. The completed atomizer assembly is then inserted into a penetration made into desired areas of the building's HVAC system. The building's HVAC system locations include, but are not limited to, the supply air ductwork, return air ductwork, and/or evaporator coil encasement. Further, the seal-ring groove is inserted into the drilled penetration hole with the ring groove on seal ring locked into the penetration hole. The tubing adapter 8 allows the connection of liquid-supply tubing to the check valve 7. The tubing adapter 8 can be provided as a two-part adapter with a male connector and a female connector. The female connector can be secured to the check valve 7, while the male connector is secured to the liquid-supply tubing. Further, the male connector and the female connector of the tubing adapter 8 can be secured by an adjustable clamp and tightened to avoid leakage of the quantity of treatment solution 10. Furthermore, tee connectors can be used to connect multiple atomizer assemblies to the same liquid-supply tubing. The liquid-supply tubing is connected to the pump outlet 4 using a pump fitting and tightly secured to avoid leakage of the quantity of treatment solution 10. In other embodiments, different fasteners and connectors can be utilized to secure the plurality of atomizer assemblies 5 to the at least one pump 2.

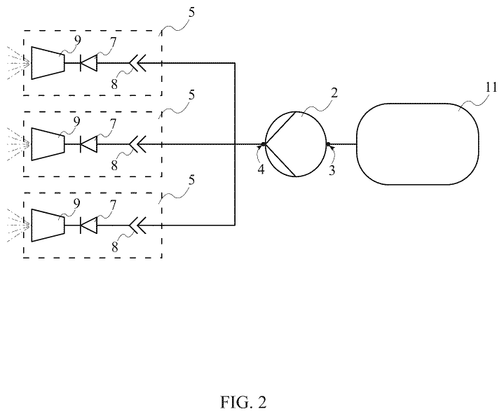

In another embodiment, the present invention can be designed to directly apply the quantity of treatment solution 10 into the target living area of the building. In this embodiment, each of the plurality of atomizer assemblies 5 may comprise a fogger nozzle 9, a check valve 7, and a tubing adapter 8, as can be seen in FIG. 2. Unlike the atomizer nozzle 6, the fogger nozzle 9 allows for a safer application of the quantity of treatment solution 10 directly into living areas where people may be residing. The fogger nozzle 9 of each of the plurality of atomizer assemblies 5 can be installed on strategic locations around the building, such as the ceiling corners of the living room, dining room, etc. Several fogger nozzle 9s can also be installed in clusters sharing a common support structure, such as a rail, to deliver the atomized quantity of treatment solution 10 to the target air-conditioned area. So, the fogger nozzle 9 is terminally connected to the check valve 7 to secure the fogger nozzle 9 to the check valve 7. The tubing adapter 8 is also terminally connected to the check valve 7, opposite to the fogger nozzle 9, to secure the tubing adapter 8 to the check valve 7. Further, the tubing adapter 8 is in fluid communication with the pump outlet 4 to allow the flow of pressurized quantity of treatment solution 10 from the at least one pump 2 to the fogger nozzle 9. This way, the quantity of treatment solution 10 can be directly applied to living areas around the building to improve the indoor air quality. In other embodiments, different dispensing nozzles can be utilized to dispense the pressurized quantity of treatment solution 10 in appropriate forms onto the target areas inside the building.

As previously discussed, the plurality of atomizer assemblies 5 can be installed in target locations around the building in order to improve the indoor air quality. In one embodiment, the present invention can be installed on specific areas throughout the building's HVAC system. For example, an atomizer assembly of the plurality of atomizer assemblies 5 can be mounted within a supply air ductwork of the HVAC system. This way, the atomized quantity of treatment solution 10 can be applied to the surfaces surrounding the supply air ductwork to eliminate bacteria, viruses, and other pollutants present in the supply air ductwork. Another atomizer assembly of the plurality of atomizer assemblies 5 can be mounted within a return air ductwork of a HVAC system. This way, the atomized quantity of treatment solution 10 can be applied to the surfaces surrounding the return air ductwork to eliminate bacteria, viruses, and other pollutants present in the return air ductwork. Further, another atomizer assembly of the plurality of atomizer assemblies 5 can be mounted within an evaporator coil encasement of a HVAC system. This way, the atomized quantity of treatment solution 10 can be applied to the surfaces surrounding the evaporator coil encasement to eliminate bacteria, viruses, and other pollutants present in the evaporator coil encasement. Thus, the present invention can provide periodic treatments to strategic locations of the HVAC system during both hot and cold modes of operation of the HVAC system.

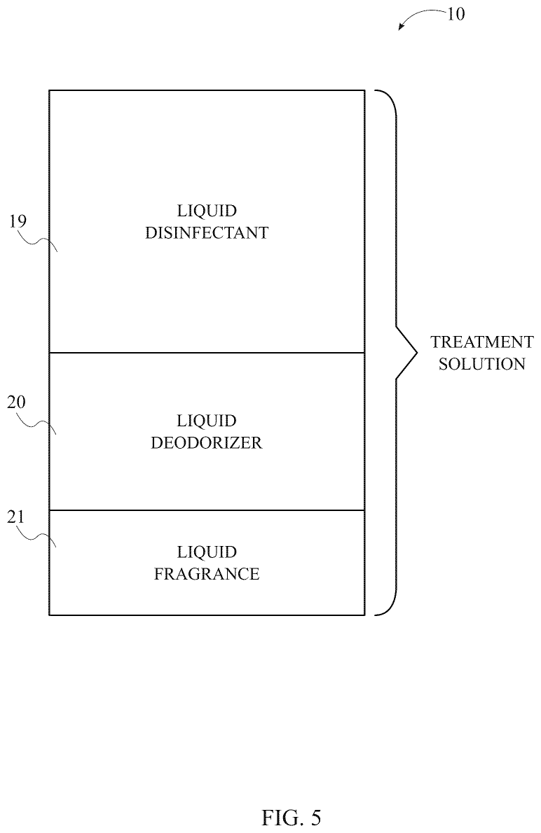

As previously discussed, the present invention allows the user to utilize different treatment solutions to be applied onto the building's target areas. As can be seen in FIG. 5, in some embodiments, the quantity of treatment solution 10 may comprise an amount of liquid disinfectant 19 that helps kill pollutants, viruses, and/or bacteria present on the target surfaces within the building. In addition, the quantity of treatment solution 10 may also comprise an amount of liquid deodorizer 20 that helps eliminates odors and other pollutants present on the target areas within the building. Further, the quantity of treatment solution 10 may also comprise an amount of liquid fragrance 21 that helps maintain a pleasant odor in the target areas within the building. Furthermore, the quantity of treatment solution 10 may include a combination of the ingredients so that the different ingredients can be dispensed together. In other embodiments, different ingredients can be included in the quantity of treatment solution 10.

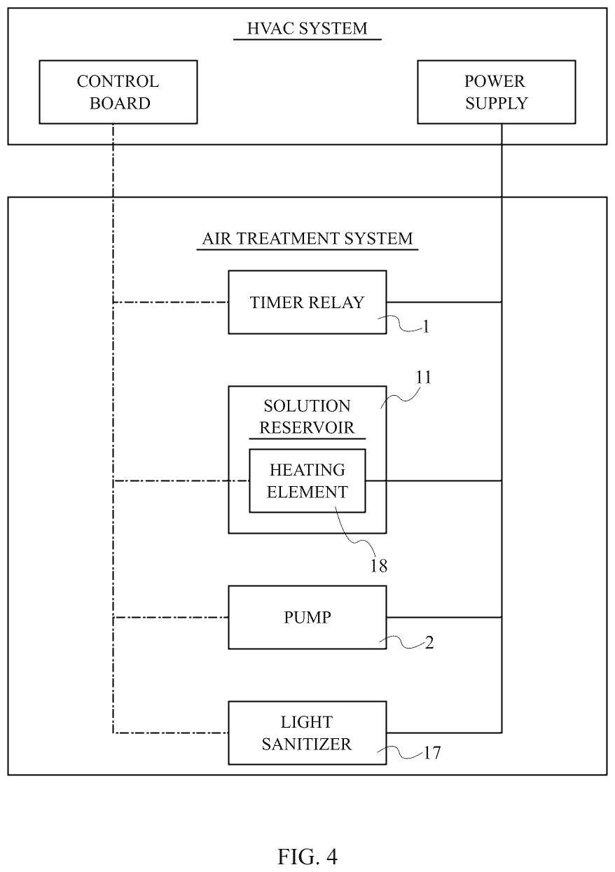

When connected to the building's HVAC system, the present invention can utilize the power supply of the HVAC system for operation. For example, as can be seen in FIG. 4, the timer relay 1 can be electronically connected to the furnace or air handler circuit board so that the operation of the timer relay 1 can follow the operation of the HVAC system. For example, the present invention can be configured to operate during the HVAC system's hot and/or cold cycles. Further, the timer relay 1 can be configured to operate independently from the HVAC system so that the present invention can periodically engage the at least one pump 2 regardless of the mode of operation of the HVAC system. Further, the timer relay 1 and the at least one pump 2 can be electrically connected to the power supply of the HVAC system to receive the current necessary for the operation of the timer relay 1 and the at least one pump 2. In other embodiments, the present invention can be controlled and/or powered using different installed systems on the building.

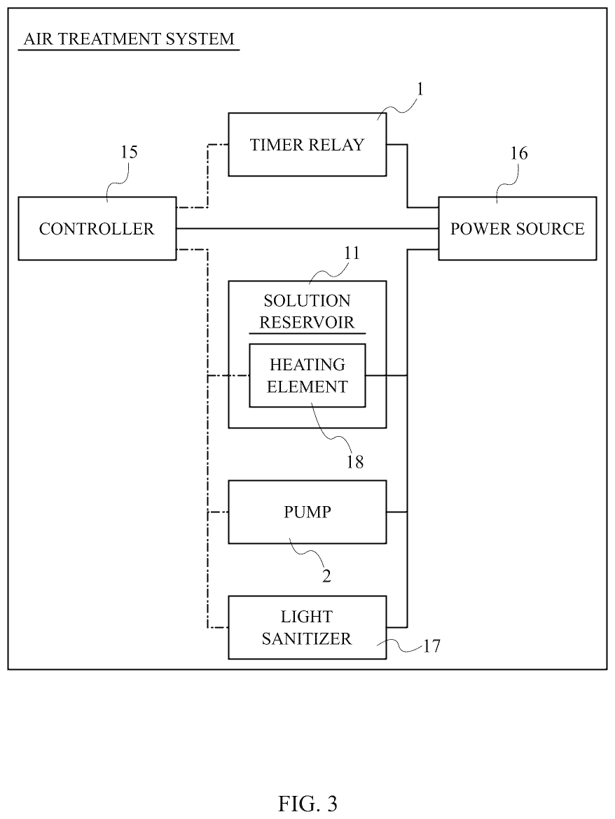

In another embodiment, the present invention may further comprise a controller 15 and a power source 16 that enables the present invention to operate independently, as can be seen in FIG. 3. The controller 15 and the power source 16 can be mounted adjacent to the at least one pump 2 or in a location that is accessible to the user for easy control of the present invention. Further, the controller 15 and the power source 16 can be provided within a housing that also retains the rest of the components of the present invention so the present invention can be easily installed on the building. Further, the controller 15 is electronically connected to the at least one pump 2 and the timer relay 1 so that timer relay 1 can receive command signals from the controller 15. The controller 15 may provide a user interface that allows the user to directly configure the settings of the present invention. The controller 15 may also include a wireless module that allows the user to remotely configure the present invention from a remote control. Further, the power source 16 is electrically connected to the controller 15, the timer relay 1, and the at least one pump 2 to provide the current necessary for the operation of each device. The power source 16 can be a battery power source 16 that can be recharged or replaced for continuous operation of the present invention. Alternatively, the power source 16 can be designed to enable the connection of the present invention to external sources such as the building's electric utilities.

In some embodiments, the present invention can provide non-chemical means of sanitizing surfaces and objects around the target areas. For example, the present invention may comprise at least one light sanitizer 17 that allows the sanitation of surfaces within the target building location. As can be seen in FIGS. 3 and 4, the at least one light sanitizer 17 is electronically connected to the controller 15 so that the operation of the at least one light sanitizer 17 can be controlled by the user via the controller 15. Like the at least one pump 2, the at least one light sanitizer 17 can be configured to periodically operate according to predetermined settings set by the user. The at least one light sanitizer 17 can also be selectively engaged via the controller 15 or can be configured to operate according to external signals, such as the activation of the HVAC system or other external stimuli. Further, the at least one light sanitizer 17 is electrically connected to the power source 16 to receive the current necessary for the operation according to the predetermined settings. In other embodiments, different cleaning, sanitating, and/or deodorizing devices can be implemented with the present invention.

In embodiments where the present invention is configured to dispense fragrances, the present invention can include means to facilitate the dispensing of the fragrances. As can be seen in FIG. 1 through 9, the present invention may further comprise at least one heating element 18 that heats the fragrances included in the quantity of treatment solution 10 to improve the fragrance dispensing. To do so, the at least one heating element 18 is mounted within the solution reservoir 11 so that the fragrances in the quantity of treatment solution 10 are heated by the at least one heating element 18. Further, the at least one heating element 18 is electronically connected to the controller 15 so that the operation of the at least one heating element 18 is controlled by the user via the controller 15. In addition, the user can configure the operation of the at least one heating element 18 via the controller 15 so that the at least one heating element 18 operates under predetermined conditions. Furthermore, the at least one heating element 18 is electrically connected to the power source 16 to receive the current necessary for the operation of the at least one heating element 18. In other embodiments, different heating devices can be implemented to improve the dispensing of the fragrances or other ingredients of the quantity of treatment solution 10.

In some embodiments, the quantity of treatment solution 10 can be maintained in the solution container that the quantity of treatment solution 10 was originally provided. A cap seal and barb fitting can be connected to the opening of the solution container and liquid-supply tubing to allow the flow of the quantity of treatment solution 10 from the solution container to the pump inlet 3. Further, the solution container is preferably mounted in a vertical orientation with the container opening oriented towards the ground to facilitate the flow of the quantity of treatment solution 10 from the solution container to the pump inlet 3 due to gravity. In other embodiments, the quantity of treatment solution 10 is poured from the solution container into the solution reservoir 11 so that the quantity of treatment solution 10 can be dispensed from the solution reservoir 11. As can be seen in FIG. 1 through 9, the solution reservoir 11 may comprise a reservoir body 12, a reservoir outlet 13, and a reservoir fitting 14. The reservoir body 12 corresponds to the structure that retains the quantity of treatment solution 10. The reservoir outlet 13 corresponds to the opening of the reservoir body 12 that allows the outflow of the quantity of treatment solution 10 from the reservoir body 12. The reservoir fitting 14 allows the connection of the liquid-supply tubing to the reservoir body 12 to allow the quantity of treatment solution 10 to flow towards the pump inlet 3. So, the reservoir outlet 13 is externally integrated into the reservoir body 12 to allow for the quantity of treatment solution 10 to flow through the reservoir body 12. Further, the reservoir outlet 13 is in fluid communication with the pump inlet 3 by the reservoir fitting 14. Furthermore, like the solution container, the reservoir body 12 can be oriented vertically so that the reservoir outlet 13 is oriented towards the ground to enable gravity-feeding of the quantity of treatment solution 10. In other embodiments, different feeding mechanisms can be utilized to supply the quantity of treatment solution 10 to the at least one pump 2.

Although the invention has been explained in relation to its preferred embodiment, it is to be understood that many other possible modifications and variations can be made without departing from the spirit and scope of the invention.

1-20. (canceled)

21. An air treatment system comprising:
a timer relay;
at least one pump;
a power source;
a plurality of atomizer assemblies each comprising an atomizer nozzle, a check valve and a tubing adapter;
a plastic ring grooved locking seal cap engaged with the tubing adapter;
a solution reservoir with a heating element;
at least one pump comprising a pump inlet and a pump outlet;
a quantity of solution being retained within the solution reservoir;
solution reservoir being in fluid communication with the pump inlet;
each of the plurality of atomizer assemblies being in fluid communication with the pump outlet and mounted to a supply air ductwork of a heating, ventilation and air conditioning (HVAC) system or the return air ductwork of a heating, ventilation and air conditioning (HVAC) system;
the timer relay being electronically connected to the at least one pump and configured to operate independently of the HVAC system; and
a controller with a user interface that controls the at least one pump, the timer relay and the heating element wherein the power source is electrically connected to the controller

22. The air treatment system as claimed in claim 21, comprising:
each atomizer nozzle being terminally connected to a check valve;
each tubing adapter also being terminally connected to the check valve, opposite the atomizer nozzle; and each tubing adapter being in fluid communication with the pump outlet.

23. The air treatment system as claimed in claim 21, comprising:
wherein the quantity of treatment solution comprises an amount of liquid disinfectant.

24. The air treatment system as claimed in claim 21, comprising:
wherein the quantity of treatment solution comprises an amount of liquid deodorizer.

25. The air treatment system as claimed in claim 21, comprising:
wherein the quantity of treatment solution comprises an amount of liquid fragrance.

26. The air treatment system as claimed in claim 21, comprising:
wherein the quantity of treatment solution comprises a liquid disinfectant without a deodorizer.

27. The air treatment system as claimed in claim 21, comprising:
at least one light sanitizer;
the at least one light sanitizer being positioned offset to the at least one pump; the at least one light sanitizer being electronically connected to the controller; and the at least one light sanitizer being electronically connected to the power source.

28. The air treatment system as claimed in claim 21, comprising:
the solution reservoir comprising a reservoir body, a reservoir outlet, and a reservoir fitting; the reservoir outlet being externally integrated into the reservoir body; the reservoir outlet being in fluid communication with the pump inlet.

29. (canceled)

30. (canceled)

31. An air treatment system comprising:
a timer relay;
at least one pump;
a plurality of atomizer assemblies;
a quantity of treatment solution;
a solution container;
a controller for the at least one pump and the timer relay that includes a wireless module to configure from a remote control;
a power source;
wherein the power source is a battery;
the at least one pump comprising a pump inlet and a pump outlet;
the quantity of treatment solution being retained within the solution container;
the solution container mounted in a vertical orientation being in fluid communication with the pump inlet;
each of the plurality of atomizer assemblies being in fluid communication with the pump outlet;
the controller being electronically connected to the at least one pump and the timer relay;
a cap seal and barb fitting connected to an opening of the solution container; and
the power source being electrically connected to the controller, the timer relay, and the at least one pump; and the atomizer assemblies are also mounted within an evaporator coil encasement of a HVAC system.

32. (canceled)

33. (canceled)

34. The air treatment system as claimed in claim 31, wherein the quantity of treatment solution comprises an amount of liquid disinfectant, an amount of liquid deodorizer, and an amount of liquid fragrance.

35. The air treatment system as claimed in claim 31, comprising:
at least one light sanitizer;
at least one heating element;
the at least one light sanitizer being positioned offset to the at least one pump;
the at least one heating element being mounted within the solution reservoir;
the controller being electronically connected to the at least one light sanitizer and the at least one heating element; and
the power source being electrically connected to the at least one light sanitizer and the at least one heating element.

36. (canceled)

37. The air treatment system as claimed in claim 31, wherein the battery is rechargeable.

38. The air treatment system as claimed in claim 31, wherein the controller also controls the heating element.
